(19) United States
(12) Patent Application Publication
(10) Pub. No.: US 2021/0205595 A1
(43) Pub. Date: Jul. 8, 2021

(54) FLUID COUPLING

(71) Applicant: PALMAYA PTY LTD., Subiaco (AU)

(72) Inventor: Alexander George Brian O'NEIL, Subiaco (AU)

(21) Appl. No.: 17/059,542

(22) PCT Filed: May 30, 2019
(86) PCT No.: PCT/AU2019/050551

§ 371 Date: Nov. 30, 2020

(30) Foreign Application Priority Data
May 30, 2018 (AU) 2018901915

(51) Int. Cl. A61M 39/10

(52) U.S. Cl. CPC A61M 39/105; A61M 2039/242; A61M 2039/1077

(57) ABSTRACT

A fluid coupling (10) comprising several intravenous fluid delivery tubes (11) and a connector (15). The connector (15) is configured for attachment to an intravenous cannula or needle. The connector (15) comprises a body (20) having a first end portion (21) and a second end portion (22). The first end portion (21) comprises a plurality of sockets (23), each socket being adapted to receive an end section of a respective fluid delivery tube (11). The second end portion (22) comprises a nozzle (31) having an end face (33), wherein there is a plurality of interior flow paths (41) within the nozzle. Each flow path (41) opens onto the end of the nozzle (31) and communicates with a respective one of the sockets (23), whereby fluid delivered by each tube (11) is transported along the respective flow path (41) to the end of the nozzle (31) and the intravenous cannula or needle attached thereto. Each tube (11) has an end section thereof sealingly received in a respective socket (23).

### TECHNICAL FIELD

This invention relates to a fluid coupling for connecting a plurality of fluid sources to an outlet member, and to a connector for use as part of a fluid coupling.

The invention is particularly applicable as a coupling for joining several intravenous fluid delivery tubes to a single-bore outlet member (such as an intravenous cannula or needle). Accordingly, it will be convenient to hereinafter disclose the invention in relation to that exemplary application. However, it is to be appreciated that the invention is not limited to that application and may be used in other applications requiring connection of a plurality of fluid sources to a single-bore outlet member of some form.

### BACKGROUND ART

The following discussion of the background art is intended to facilitate an understanding of the present invention only. The discussion is not an acknowledgement or admission that any of the material referred to is or was part of the common general knowledge as at the priority date of the application.

In medical treatment of patients, it is often necessary to administer fluids parenterally. In certain circumstances, several fluids are administered, possibly over varying lengths of time. More particularly, it is common to provide intravenous drug delivery using multiple infusions delivered through a single intravenous cannula. By way of example, the patient may be continuously supplied with one fluid, such as a saline solution, and intermittently injected with one or more other fluids, all through a single cannula or needle. Accordingly, there is a need to connect the cannula or needle to several different fluid sources.

There have been various proposals for fluid couplings to provide such a connection. Some such proposals have, however, proved to be problematic, as a result of causing unacceptable variations in the flow rates of the different fluids. This is particularly so in the case of fluid couplings in which there is a relatively large common space in which the different fluids from the tubes mix prior to entering the cannula or needle. The existence of a relatively large common space in which prior mixing of the fluids occurs may result one of the fluids affecting the flow rate of another fluid, leading to abnormalities in the intended administration of the fluid to the patient.

In international application WO 91/10861 there is disclosed a fluid coupling for coupling several fluid delivery tubes to a single intravenous cannula or needle, which seeks to avoid a relatively large common space in which the different fluids from the tubes mix prior to entering the cannula or needle. In the arrangement disclosed in WO 91/1086, which has become known as a “V-set”, there are at least three tubes having outlet ends received within a connector to which the cannula or needle is adapted to be attached. The outlet ends of the tubes are received within a cavity extending through the connector and are bonded in position. The connector is configured as a male luer connector adapted for connection to a counterpart female luer connector to which the cannula or needle is attached. The three tubes extend to the end of the male connector which will be contiguous with the cannula or needle when the female connector is coupled to the male connector. This arrangement ensures that there is no common space, or at least minimal common space, through which fluid discharging from each tube passes before entering the bore of the cannula or needle. In this way there is no, or at least minimal, mixing of the fluids prior to entry into the cannula or needle, which might otherwise cause variations in the flow rates of the respective fluids (as discussed in WO 91/10861, contents of which are incorporated herein by way of reference).

While the V-set fluid coupling disclosed in WO 91/10861 has proved to be a commercial success, it has certain deficiencies. Firstly, the V-set fluid coupling can be somewhat time consuming to fabricate. This is because it is necessary to feed the tubes through the cavity within the male luer, and then bond them into position using a suitable adhesive or resin. Once the adhesive or resin has cured, the projecting tube ends, and any excess adhesive or resin, are severed at the end of the connector to leave a flush connector end. Further, this process of fabrication may bring about certain deficiencies in operation of the V-set as a fluid coupling. In particular, it is likely that the end section of each tube would have experienced some stretching in the process of being fed into the cavity, leading to a reduction the cross-section flow area of the lumen and thus restricting flow. This stretching can also contribute to degradation of any internal coating provided within the tubing to resist drug sorption. Furthermore, there are space constraints arising from the need to feed a plurality of tubes through a single cavity and have sufficient space remaining to receive the adhesive or resin. Because of these space constraints, there are limitations regarding the cross-sectional size of tube that can be used to fabricate the V-set fluid coupling.

It is against this background, and the problems and deficiencies associated therewith, that the present invention has been developed.

### SUMMARY OF INVENTION

According to a first aspect of the invention, there is provided a connector comprising a body having a first end portion and a second end portion, the first end portion comprising a plurality of sockets, each socket being adapted to receive an end section of a respective fluid delivery tube, and the second end portion comprising a nozzle having an end, wherein there is a plurality of interior flow paths within the nozzle, each flow path opening onto the end of the nozzle, and wherein each flow path communicates with a respective one of the sockets whereby fluid delivered by each tube is transported along the respective flow path to the end of the nozzle.

With this arrangement, there is no common space within the body within which fluids delivered by the tubes can mix prior to discharging from the nozzle.

Preferably, the body is formed by injection moulding a plastics material, although of course may be formed in any other appropriate way (such as for example by additive manufacturing).

Preferably, the nozzle presents an end face onto which the flow paths open.

The connector may be configured as a male luer connector. The male luer connector may be adapted to be coupled to a matching female luer connector to which a cannula or needle may be attached. This arrangement facilitates continuous flow of fluid from the lumen of each tube, along the respective flow path within the body, and into the female luer connector, without any prior mixing with fluids delivered by the other tubes.

The flow paths may be of cross-sectional profiles configured to facilitate a compact arrangement of the flow paths within the confines of the nozzle.

The flow paths may be defined by passages, wherein each passage may comprise a curved periphery having an outer side section, an inner side section and two opposed end sections, and wherein the outer side sections are located on a generally common circumference and the inner side sections are disposed inwardly of said circumference.

The flow paths may be defined by passages, wherein the passages are each of circular cross-section and disposed in circumferentially spaced relation within the nozzle.

According to a second aspect of the invention there is provided a fluid coupling comprising a connector according to the first aspect of the invention and a plurality of tubes, wherein each tube has an end section sealingly received in a respective socket.

Each tube end section is sealing received in the respective socket in the sense that a sealed (fluid-tight) connection is established between the tube and the socket. The sealed connection may be established in any appropriate way, such as by bonding the tube end section in the socket or by way of an interference fit of the tube end section in the socket.

The tubes are adapted to receive fluids for delivery to a patient via a common outlet member, such as a cannula or needle coupled to the nozzle. Where the connector comprises a male luer, the common cannula or needle may be attached to a counterpart female luer adapted for mating connection with the male luer.

The tubes may be configured for several different fluid delivery regimes. By way of example, one tube may comprise a conduit for delivery of fluid from a gravity-fed source. Another tube may comprise a conduit for delivery of fluid from a pressurised source (such as an infusion pump). Yet another tube may comprise a conduit configured to allow intermittent injections or bolus infusions to be delivered through a syringe or line for bolus injections. Yet another tube may comprise a conduit configured to facilitate constant infusions.

The fluid coupling would have at least two tubes and typically three or more. The tubes may comprise any combination of tubes described above, and optionally also other tubes.

One or more of the tubes may each be fitted with a valve or coupling element to facilitate easy attachment and detachment of fluid sources and ancillary components (as the case may be). Where a valve or coupling element is provided, it may comprise a sealable valve (such as for example a swab-able valve). Typically, all tubes would be fitted with such valves or coupling elements.

One or more of the tubes may each incorporate, be connected to, or be otherwise associated with a non-return valve. The non-return valve may be integrated in a non-return valve set associated with the respective tube. By way of example, a non-return valve may be provided to prevent reverse flow of fluid into a fluid source in the event of a pressure surge in one of the other tubes. Typically, a high flow tube (e.g. one receiving fluid from a gravity-fed source) would require a relatively low pressure opening valve. Other tubes may require a relatively high pressure opening valve.

In an arrangement in which the valve or coupling element comprises a sealable valve, there may be a non-return valve associated with the sealable valve.

One or more of the tubes may each incorporate, or be connected to, or be otherwise associated with a filter (such as for example a bacterial filter).

One or more of the tubes may each be lined with an internal coating resistant to drug sorption; the coating may, for example, comprise polyethylene.

One or more of the tubes may each be fitted with a guard along at least a portion of the length of the tube to resist over-bending and kinking. The guard may comprise a sleeve positioned on the exterior of the tube. The sleeve may comprise a helical or coiled spring. This is likely to be particularly suitable for a relatively large-bore conduit receiving fluid from a gravity-fed source. The sleeve positioned on the exterior of the tube may offer lateral reinforcement to the tube. This may permit use of thin-walled tubing that would not in itself be dimensionally stable. The sleeve may extend along substantially the full length of the respective tube. Further, the sleeve may be anchored at its ends to the tube or to fittings attached to the tube.

According to a third aspect of the invention there is provide a fluid coupling comprising: a connector and a plurality of tubes; wherein the connector comprises a body having a first end portion and a second end portion, the first end portion comprising a plurality of sockets, each socket being adapted to receive an end section of a respective fluid delivery tube, and the second end portion comprising a nozzle having an end, wherein there is a plurality of interior flow paths within the nozzle, each flow path opening onto the end of the nozzle; and wherein each flow path communicates with a respective one of the sockets whereby fluid delivered by each tube is transported along the respective flow path to the end of the nozzle, and wherein each tube has an end section thereof sealingly received in a respective socket.

The fluid coupling according to the third aspect of the invention may optionally have any one or more features of the fluid coupling according to the second aspect of the invention.

According to a fourth aspect of the invention there is provide a fluid coupling comprising a connector, a plurality of tubes each having one end coupled to the connector and another end coupled to a respective fitting, wherein at least one of the tubes has a guard along at least a portion of the length thereof.

The guard may comprise a sleeve positioned on the exterior of the tube. The sleeve may comprise a helical or coiled spring. This is likely to be particularly suitable where the tube comprises a relatively large-bore conduit receiving fluid from a gravity-fed source. The sleeve positioned on the exterior of the tube may offer lateral reinforcement to the tube. This may permit use of thin-walled tubing that would not in itself be dimensionally stable. The sleeve may extend along substantially the full length of the respective tube. Further, the sleeve may be anchored at its ends to the tube or to the connector and fitting respectively at the ends of the tube.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a schematic view of a first embodiment of a fluid coupling according to the invention;

FIG. 2 is a sectional view of the arrangement shown in FIG. 1;

FIG. 3 is a perspective view of a connector forming part of the fluid coupling;

FIG. 4 is a side view of the connector;

FIG. 5 is a view of one end of the connector;

FIG. 6 is a view of the other end of the connector;

FIG. 7 is a section on line C-C of FIG. 6;

FIG. 8 is a section on line D-D of FIG. 6;

FIG. 9 is a perspective view of the connector from one end thereof;

FIG. 10 is a perspective view of the connector from the other end thereof;

FIG. 11 is an end view of the connector;

FIG. 12 is a view of an end of a connector for a second embodiment of a fluid coupling according to the invention;

FIG. 13 is a section on line C-C of FIG. 12;

FIG. 14 is a section on line D-D of FIG. 12;

FIG. 15 is a perspective view of the connector of FIG. 12 seen from one end thereof;

FIG. 16 is a perspective view of the connector from the other end thereof;

FIG. 17 is an end view of the connector;

FIG. 18 is a perspective view a connector for a third embodiment of a fluid coupling according to the invention, the connector being seen from one end thereof;

FIG. 19 is a perspective view of the connector of FIG. 18 from the other end thereof;

FIG. 20 is an end view of the connector;

FIG. 21 is a perspective view a connector for a fourth embodiment of a fluid coupling according to the invention, the connector being seen from one end thereof;

FIG. 22 is a perspective view of the connector of FIG. 21 from the other end thereof;

FIG. 23 is an end view of the connector; and

FIG. 24 is a schematic view of a fifth embodiment of a fluid coupling according to the invention.

The drawings shown are not necessarily to scale, with emphasis instead generally being placed upon illustrating the principles of the present invention.

The figures depict several embodiments of the invention. The embodiments illustrate a certain configuration; however, it is to be appreciated that the invention can be implemented by way of many different configurations, as would be obvious to a person skilled in the art, whilst still embodying the present invention. These configurations are to be considered within the scope of this invention.

### DESCRIPTION OF EMBODIMENTS

In the following detailed description, the present invention is described in connection with several preferred embodiments. However, to the extent that the following description is specific to particular embodiments or a particular use of the present techniques, it is intended to be illustrative only and merely provides a concise description of the exemplary embodiment. Accordingly, the present invention is not limited to the specific embodiments described below, but rather the invention includes all alternatives, modifications, and equivalents falling within the true scope of the appended claims.

The embodiments will be described with reference to a fluid coupling 10 for joining several intravenous fluid delivery tubes 11 to a single-bore outlet member in the form of an intravenous cannula (not shown). There are at least two tubes 11, as will be discussed further later.

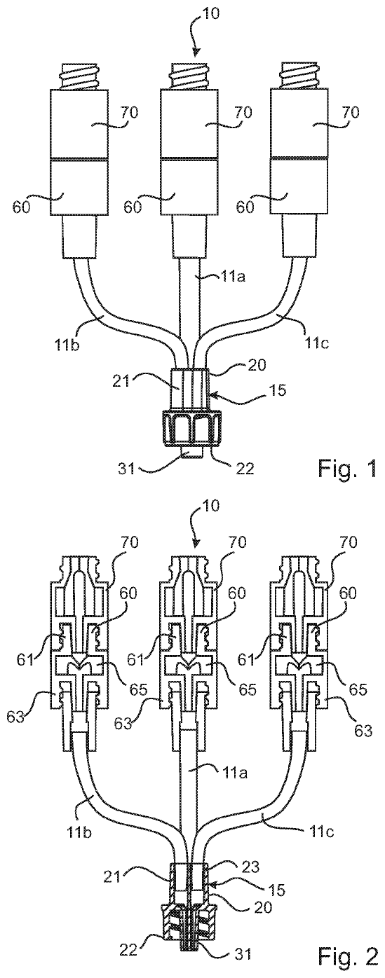

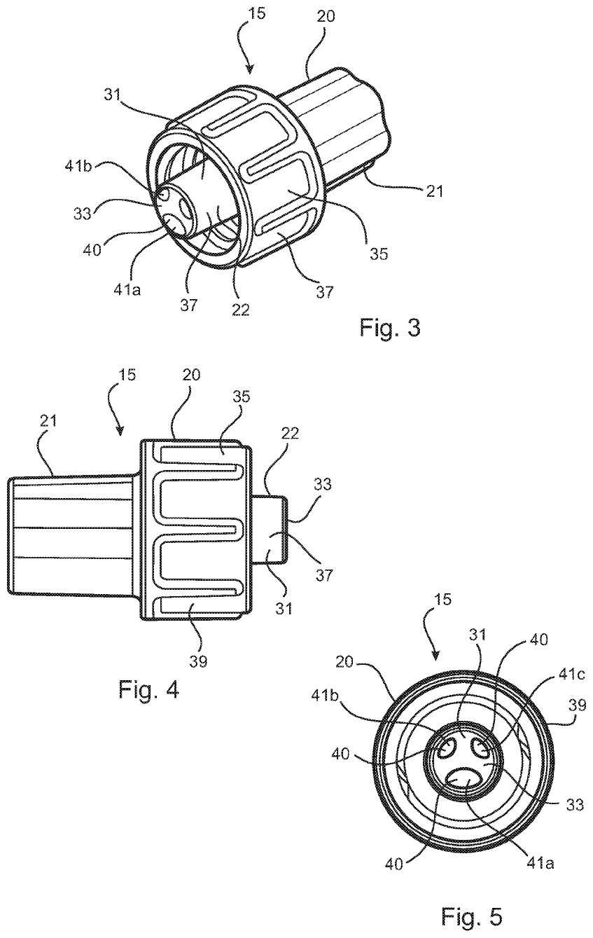

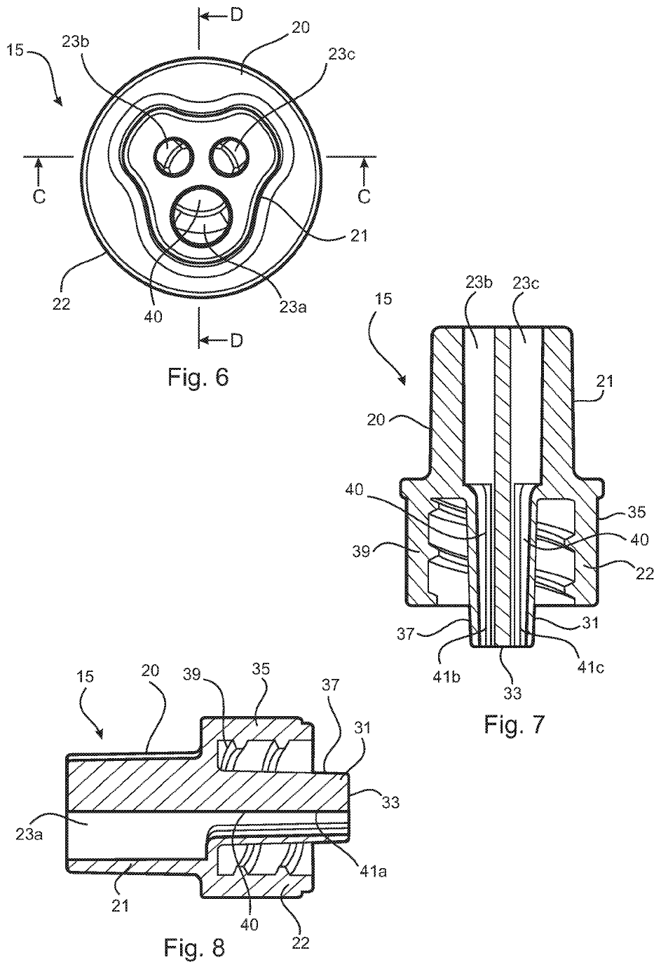

Referring to FIGS. 1 to 11, a first embodiment of the fluid coupling 10 comprises three fluid delivery tubes 11a, 11 b and 11c. Typically, the tubes 11 comprise PVC tubing, although they may be formed of any other appropriate material. One or more of the tubes 11 may be lined internally with a coating (not shown) such as polyethylene to inhibit drug sorption.

The fluid coupling 10 further comprises a connector 15. The connector 15 comprises a body 20 formed of injection moulded plastics material. The plastics material may, for example, comprise material used in the manufacture of conventional luer lock fittings for medical devices.

The body 20 has a first end portion 21 and a second end portion 22.

The first end portion 21 comprises a plurality of sockets 23, each socket being adapted to receive an end section of a respective fluid delivery tube 11. Accordingly, in the arrangement shown there are three sockets 23a, 23b and 23c corresponding to the three tubes 11a, 11 b and 11c. The end sections of the tubes 11 are sealingly received in the sockets 23 to establish a sealed (fluid-tight) connection therebetween. The sealed connection may be established in any appropriate way, such as by bonding the end sections of the tube 11 in the socket 23 or by way of an interference fit between the end sections of the tubes 11 and the sockets 23.

The second end portion 22 comprises a nozzle 31 having an end defining an end face 33. The second end portion 22 is configured as a male luer connector portion 35, wherein the nozzle 31 provides a male taper fitting 37 and an internally threaded hub 39 surrounds the male taper fitting 37.

With this arrangement, the connector 15 is configured as a male luer connector adapted for connection to a matching female luer connector (not shown) to which the intravenous cannula is attached. The female luer connector and the attached intravenous cannula may be of known kind.

Within the nozzle 31 there is a plurality of passages 40 defining interior flow paths 41, each flow path 41 opening onto the end face 33 of the nozzle 31. Each flow path 41 communicates with a respective one of the sockets 23, whereby fluid delivered by each tube 11 is transported along the respective flow path 41 to the end 33 of the nozzle 31. Accordingly, in the arrangement shown there are three flow paths 41a, 41b and 41c which correspond to the three sockets 23a, 23b and 23c.

With this arrangement, there is no common space within the body 20 within which fluids delivered by the tubes 11 can mix prior to discharging from the nozzle 31. Upon discharging from the nozzle 31 via flow paths 41, fluids delivered by the tubes 11 immediately enter the female luer connector and the intravenous cannula. This arrangement facilitates continuous flow of fluid from the lumen of each tube 11, along the respective flow path 41 within the nozzle 31, and into the female luer connector, without any prior mixing with fluids delivered by the other tubes.

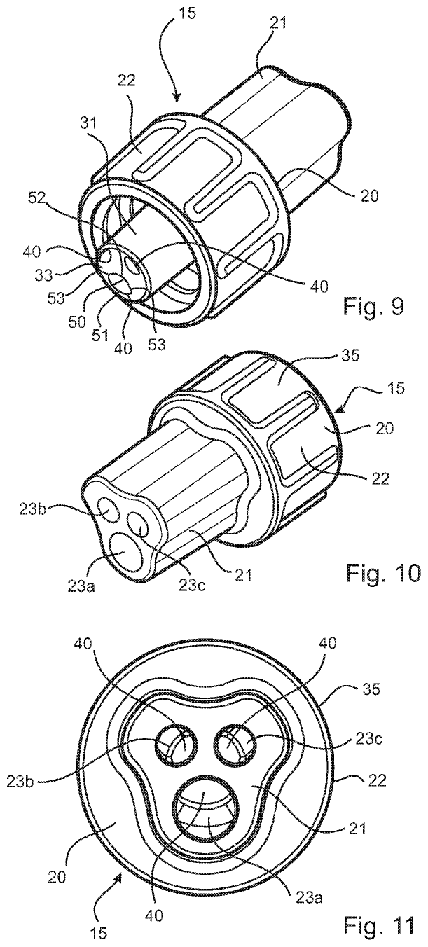

The passages 40 may be of cross-sectional profiles configured to facilitate a compact arrangement within the confines of the nozzle 31. Each passage 40 has an inner end communicating with the respective socket 23 and an outer end opening onto the end face 33 of the nozzle 31. In the arrangement shown, each passage 40 tapers inwardly from the inner end to the outer end. Further, each passage 40 has a curved periphery 50. More particularly, each passage 40 has a cross-sectional profile resembling a somewhat deformed oval shape, at least at the section thereof opening onto the end face 33 of the nozzle 31. The oval shape of the periphery 50 may be considered to comprise an outer side section 51, an inner side section 52 and two opposed end sections 53, as identified in FIG. 9. The outer side sections 51 of the three passages 40 are located on a generally common circumference (as best seen in FIG. 5), and the inner side sections 52 are disposed inwardly of that circumference. This configuration enhances use of available space within the confines of the nozzle 31 to accommodate the passages 40. Other profiles and arrangements of the passages 40 are, of course, also contemplated. By way of example, in a basic form the passages 40 may each be of circular cross-section and disposed in circumferentially spaced relation within the nozzle 31.

In this first embodiment, tube 11a comprises a relatively large-bore conduit, and tubes 11b and 11c each comprise a relatively small-bore conduit. Accordingly, the tube 11a is of larger outside diameter than tubes 11a, 11 b, and as such socket 23a is larger in cross-sectional size than sockets 23b, 23c (as can be seen in the drawings).

Each tube 11 has an opposed end section provided with a fitting 60 to facilitate coupling to a fluid source.

Preferably, the fittings 60 are permanently bonded onto the tubes 11 to ensure that they remain in place during the service life of the fluid coupling 10. Throughout the service life of the fluid coupling 10, it would likely be that the fluid coupling would be subjected to a range of stresses which might otherwise cause leakage or detachment of the fittings 60 if they were not permanently bonded onto the tubes 11. For example, it is likely that the fluid coupling 10 would be used on a multitude of occasions throughout its service life, with a range of fluid sources being connected to, and disconnected from, the tubes 11 via the fittings 60. Further, it is likely that the fluid coupling 10 would be used with patients who are mobile and thus likely to move about at times when fluids are not being administered. These various actions could subject the fluid coupling 10 to stresses which might otherwise cause leakage or detachment of the fittings 60 if they were not permanently bonded onto the tubes 11, as mentioned above.

In the arrangement shown, each fitting 60 comprises a coupling element in the form of a female luer lock connector 61. Other coupling arrangements are also contemplated, as would be understood by a person skilled in the art. By way of example, an alternative fitting 60 for one or more of the tubes 11 might comprise a needle access port or a sealable valve (such as a swab-able valve).

In the arrangement shown, each fitting 60 also comprises an integrated valve set 63 having a non-return valve 65. The non-return valve 65 is provided to prevent reverse flow of fluid into a fluid source in the event of a pressure surge in one of the other tubes 11. Typically, a high flow tube (e.g. one receiving fluid from a gravity-fed source) would require a non-return valve 65 in the form of a relatively low pressure opening valve. Other tubes may require non-return valves 65 each in the form of a relatively high pressure opening valve.

For the purpose of example, the fittings 60 are shown in FIGS. 1 and 2, connected to mating fittings 70 facilitating coupling to respective fluid sources.

In the case of one or more of the fittings 60 comprising a sealable valve, it is particularly advantageous that the fitting also have one of the non-return valves 65. Such an arrangement would likely assist in preventing leaking back of fluid upon disconnection from the sealable valve, and also combines the benefits of minimising the risk of bacterial contamination at the sealable valve as well as eliminating drug mixing between any two flow pathways connected to the intravenous cannula.

One or more of the tubes 11 may also have a filter (not shown), such as for example a bacterial filter.

One or more of the tubes 11 may each be fitted with a guard along at least a portion of the length of the tube to resist over-bending and kinking. The guard may comprise a sleeve positioned on the exterior of the respective tube 11. The sleeve can flex about is longitudinal axis to accommodate some bending of the tube 11, but acts to control the extent of bending to prevent over-bending or kinking (which might create a flow restriction adversely affecting fluid delivery along the tube). The sleeve may also afford lateral reinforcement to the tube. The sleeve may be configured as coiled or helical spring.

It has been found that the fluid coupling 10 can deliver flow rates exceeding that of the prior art V-set fluid coupling described previously. This is because the tubes 11 are received in sockets 23 which fluidly communicate with a cannular via integrated flow paths 41 within nozzle 31. This provides an arrangement without certain flow restrictions inherent in the prior art V-set fluid coupling as discussed previously, including restrictions arising from stretching of the end sections of the tubes during assembly of the prior art coupling.

In the first embodiment, there are different sizes of tubes 11. Specifically, tube 11a comprises a relatively large-bore conduit, and tubes 11b and 11c each comprise a relatively large-bore conduit. Accordingly, the sockets 23 are correspondingly of different sizes.

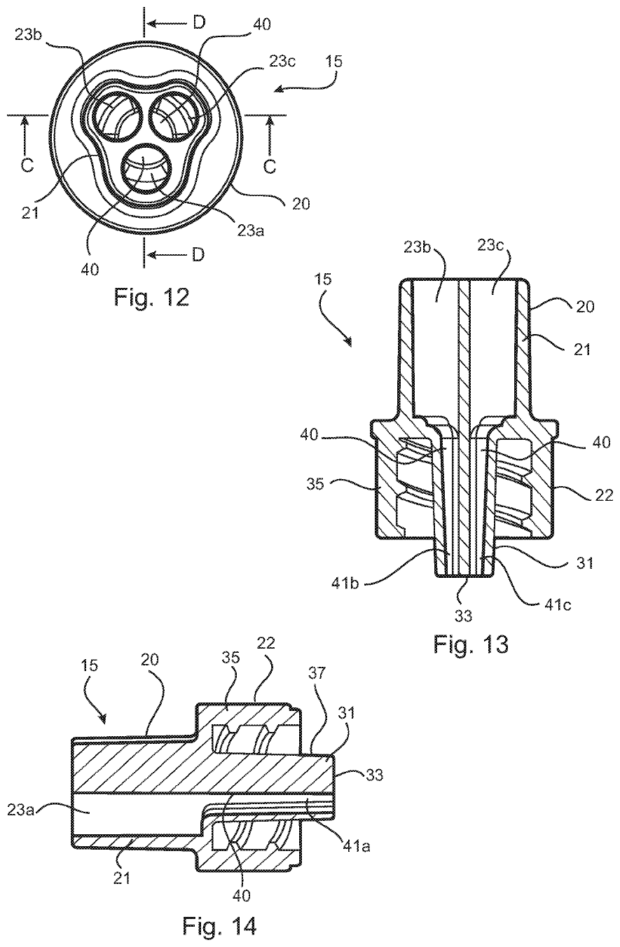

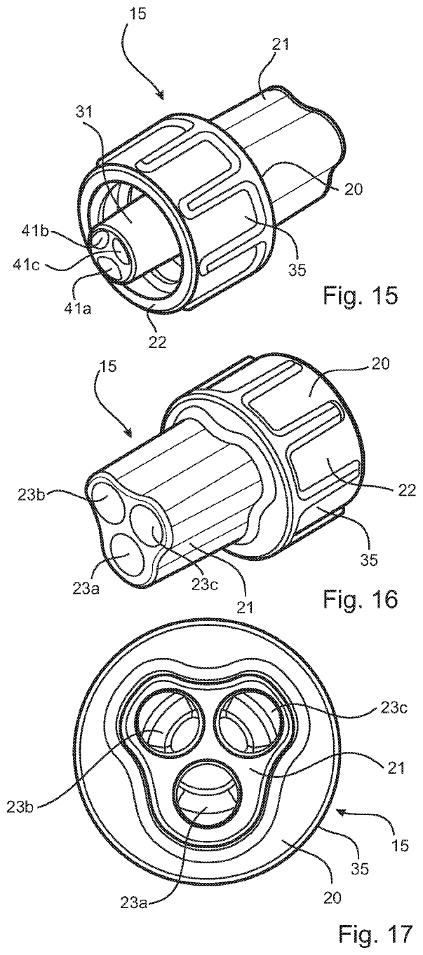

In a second embodiment, as shown in FIGS. 12 to 17, the sockets 23 are the same in size to receive tubes 11 also the same in size. Otherwise, the second embodiment is similar to the first embodiment and like reference numerals are used to identify like features.

In the first and second embodiments, the connector 15 is configured to accommodate three tubes 11. However, the invention is not limited to three tubes and any number comprising two or more tubes are contemplated.

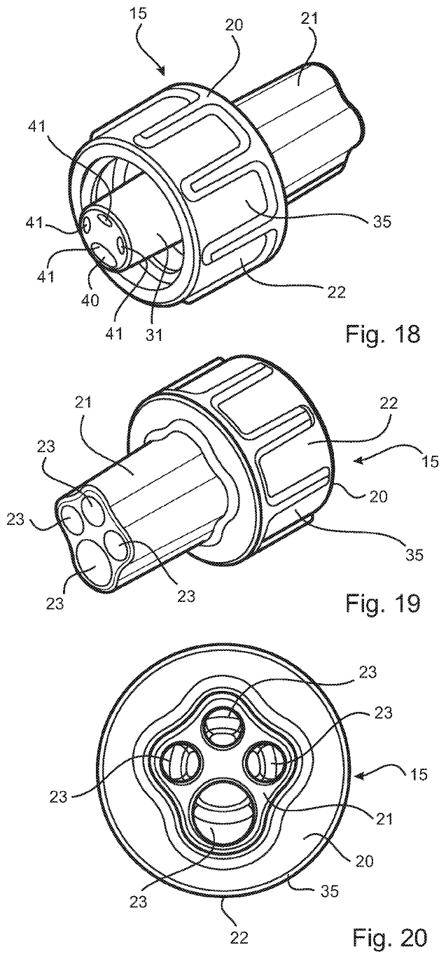

In a third embodiment, as shown in FIGS. 18 to 20, the connector 15 is configured to accommodate four tubes 11. Otherwise, the third embodiment is similar to the first embodiment and like reference numerals are used to identify like features.

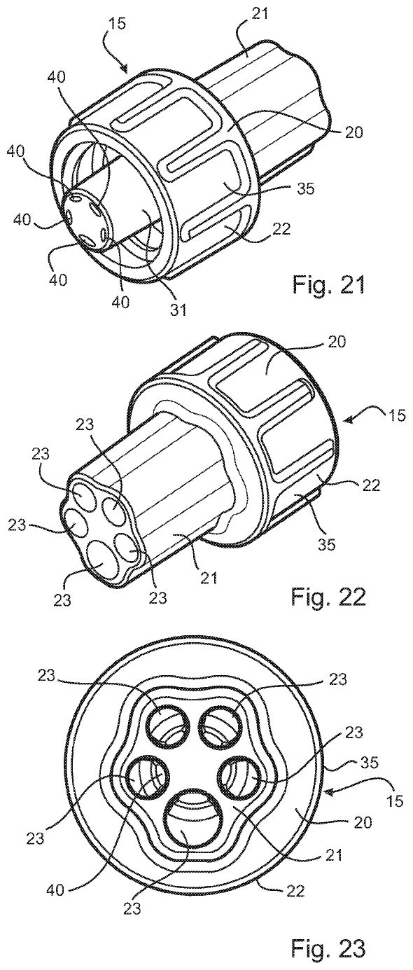

In a fourth embodiment, as shown in FIGS. 21 to 23, the connector 15 is configured to accommodate five tubes. Otherwise, the third embodiment is similar to the first embodiment and like reference numerals are used to identify like features.

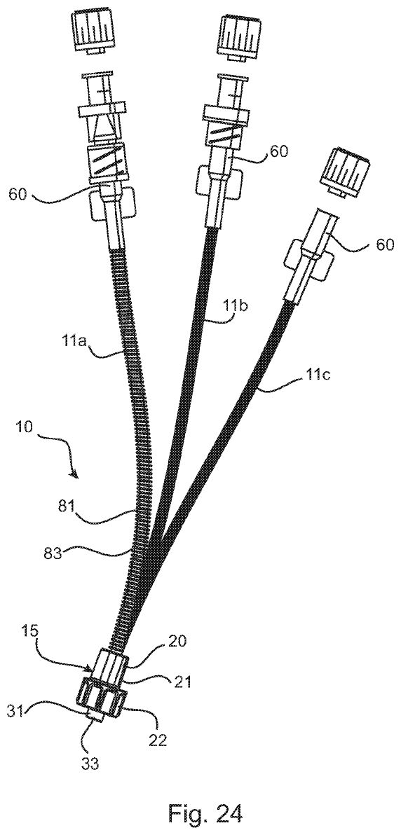

As mentioned above, one or more of the tubes 11 may each be fitted with a guard along at least a portion of the length of the respective tube(s). This is applicable to all embodiments described above. Such an arrangement is a feature of a fifth embodiment of a fluid coupling 10 according to the invention, as shown in FIG. 24. The fluid coupling 10 according to the fifth embodiment is similar in many respects to the fluid coupling according to the first embodiment and so similar reference numerals are used to identify similar parts.

Referring to FIG. 24, tube 11a is fitted with guard 81 comprising a sleeve configured as a helical or coiled spring 83 positioned on the exterior of the tube. In the arrangement shown, the spring 83 extends along substantially the full length of tube 11a and is anchored at its ends with respect to the tube. The spring 83 may, for example, be anchored at its ends with respect to the tube 11a by being connected to fitting 60 at one end and connector 15 at the other end. The spring 83 can flex about is longitudinal axis to accommodate some bending of the tube 11a, but acts to control the extent of bending to prevent over-bending or kinking (which might create a flow restriction adversely affecting fluid delivery along the tube). This is likely to be particularly suitable for a relatively large-bore conduit receiving fluid from a gravity-fed source. The spring 83 positioned on the exterior of the tube 11a affords lateral reinforcement to the tube 11a. This may permit use of thin-walled tubing that would not in itself be dimensionally stable. This maximises fluid flow, and makes the tubing thinner and more comfortable on the arm of a patient.

It should be appreciated that the scope of the invention is not limited to the scope of the embodiments described.

Further, it should be appreciated that various modifications can be made without departing from the principles of the invention. Therefore, the invention should be understood to include all such modifications within its scope.

The terminology used herein is for the purpose of describing particular example embodiments only and is not intended to be limiting.

As used herein, the singular forms “a”, “an” and “the” may be intended to include the plural forms as well, unless the context clearly indicates otherwise.

The method steps, processes, and operations described herein are not to be construed as necessarily requiring their performance in the particular order discussed or illustrated, unless specifically identified as an order of performance. It is also to be understood that additional or alternative steps may be employed.

Reference to any positional descriptions, such as “top”, “bottom” and “side”, are to be taken in context of the embodiment described and depicted in the drawings, and are not to be taken as limiting the invention to the literal interpretation of the term but rather as would be understood by the skilled addressee.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Additionally, where the terms “system”, “device”, and “apparatus” are used in the context of the invention, they are to be understood as including reference to any group of functionally related or interacting, interrelated, interdependent or associated components or elements that may be located in proximity to, separate from, integrated with, or discrete from, each other.

Throughout this specification, unless the context requires otherwise, the word “comprise” or variations such as “comprises” or “comprising”, will be understood to imply the inclusion of a stated integer or group of integers but not the exclusion of any other integer or group of integers.

1. A connector comprising a body having a first end portion and a second end portion, the first end portion comprising a plurality of sockets, each socket being adapted to receive an end section of a respective fluid delivery tube, and the second end portion comprising a nozzle having an end, wherein there is a plurality of interior flow paths within the nozzle, each flow path opening onto the end of the nozzle, and wherein each flow path communicates with a respective one of the sockets whereby fluid delivered by each tube is transported along the respective flow path to the end of the nozzle.

2. The connector according to claim 1 configured as a male luer connector.

3. The connector according to claim 1 or 2 wherein the nozzle presents an end face onto which the flow paths open.

4. The connector according to claim 1, 2 or 3 wherein the flow paths are of cross-sectional profiles configured to facilitate a compact arrangement of the flow paths within the confines of the nozzle

5. The connector according to claim 4 wherein the flow paths are defined by passages, wherein each passage comprises a curved periphery having an outer side section, an inner side section and two opposed end sections, and wherein the outer side sections are located on a generally common circumference and the inner side sections are disposed inwardly of said circumference.

6. The connector according to claim 4 wherein the flow paths are be defined by passages, wherein the passages are each of circular cross-section and disposed in circumferentially spaced relation within the nozzle.

7. A fluid coupling comprising a connector according to any one of the preceding claims and a plurality of tubes, wherein each tube has an end section sealingly received in a respective socket.

8. The fluid coupling according to claim 7 wherein the end section of each tube is sealingly received in a respective socket by bonding the tube end section in the socket or by way of an interference fit of the tube end section in the socket.

9. The fluid coupling according to claim 7 or 8 wherein the tubes are adapted to receive fluids for delivery to a patient via a common outlet member coupled to the nozzle.

10. The fluid coupling according to claim 9 wherein tubes are configured for several different fluid delivery regimes.

11. The fluid coupling according to any one of claims 7 to 10 wherein least one of the tubes is fitted with a valve or coupling element to facilitate attachment and detachment of a fluid source or ancillary component.

12. The fluid coupling according to any one of claims 7 to 11 wherein at least one of the tubes incorporates, is connected to, or is otherwise associated with a non-return valve.

13. The fluid coupling according to claim 11 wherein the valve or coupling element comprises a sealable valve and wherein there is a non-return valve associated with the sealable valve.

14. The fluid coupling according to any one of claims 7 to 13 wherein at least one of the tubes incorporates, is connected to, or is otherwise associated with a filter.

15. The fluid coupling according to any one of claims 7 to 14 wherein at least one of the tubes is lined with an internal coating resistant to drug sorption.

16. The fluid coupling according to any one of claims 7 to 15 wherein at least one of the tubes is fitted with a guard along at least a portion of the length of the tube to resist over-bending and kinking.

17. The fluid coupling according to claim 16 wherein the guard comprises a sleeve positioned on the exterior of the tube.

18. The fluid coupling according to claim 17 wherein the sleeve extends substantially the full length of the tube.

19. The fluid coupling according to claim 17 or 18 wherein the sleeve is anchored at its ends to the tube or to fittings attached to the tube.

20. The fluid coupling according to claim 17, 18 or 19 wherein the sleeve comprises a coiled or helical spring.

21. A fluid coupling comprising: a connector and a plurality of tubes; wherein the connector comprises a body having a first end portion and a second end portion, the first end portion comprising a plurality of sockets, each socket being adapted to receive an end section of a respective fluid delivery tube, and the second end portion comprising a nozzle having an end, wherein there is a plurality of interior flow paths within the nozzle, each flow path opening onto the end of the nozzle; and wherein each flow path communicates with a respective one of the sockets whereby fluid delivered by each tube is transported along the respective flow path to the end of the nozzle, and wherein each tube has an end section thereof sealingly received in a respective socket.
